(19) United States
(12) Patent Application Publication
(10) Pub. No.: US 2021/0156855 A1
(43) Pub. Date: May 27, 2021

(54) METHODS AND APPARATUS FOR SAMPLE ANALYSIS USING LATERAL FLOW

(71) Applicant: Ellume Limited, East Brisbane, Queensland (AU)

(72) Inventors: Andrea Ranzoni, East Brisbane, Queensland (AU); Sean Andrew Parsons, East Brisbane, Queensland (AU); Scott Robert Fry, East Brisbane, Queensland (AU); Christopher Robert Miller, East Brisbane, Queensland (AU)

(21) Appl. No.: 16/625,615

(22) PCT Filed: Jul. 3, 2018
(86) PCT No.: PCT/AU2018/050687

§ 371 Date: Dec. 20, 2019

(30) Foreign Application Priority Data
Jul. 4, 2017 (AU) 2017902606

(51) Int. Cl. G01N 33/558; G01N 21/64; G01N 33/543

(52) U.S. Cl. CPC G01N 33/558; G01N 2021/6441; G01N 33/54386; G01N 21/6428

(57) ABSTRACT

Methods and assays for performing a lateral flow test are disclosed. A sample is applied to a receiving portion of a lateral flow device such that the sample flows to at least a first test zone and a second test zone. First and second signals levels at the first and second test zones over an assay period are monitored. If a first analyte of interest is present in the sample, the first analyte is labelled and the presence of labelled first analyte in the sample causes one of the first and second signals levels to increase during the assay period. A change between the first and second signal levels over a period of time during the assay period is monitored. The sample can be incubated prior to application to the receiving portion to provide homogeneity to labelling and therefore a substantially linear increase of the signal level at one of the test zones and a substantially constant signal level at the other one of the test zones.

### CROSS-REFERENCE TO RELATED APPLICATION

The present application claims priority to Australian provisional patent application no. 2017902606 filed 4 Jul. 2017, the entire content of which is hereby incorporated by reference.

### TECHNICAL FIELD

The present disclosure relates to methods and apparatus for making determinations about one or more target analytes and/or medical conditions in a human or animal body. For example, the present disclosure relates to methods and apparatus for making determinations about one or more target analytes and/or medical conditions, based on a sample received from a human or animal body, using a lateral flow assay.

### BACKGROUND

Lateral Flow Assays (LFAs) have been in the in vitro diagnostics market for over 25 years and are widely regarded as inexpensive, easy to use, rapid and qualitative tests that can be used in point-of-care or field-based settings.

LFAs exploit the migration of a liquid sample along a porous membrane material such as nitrocellulose. Capture and detection of one or more target analytes takes place as the sample flows across discrete zones or lines immobilised with a capture reagent. Various capture reagents can be used, though antibodies are often the preferred choice. LFAs in which antibodies are used are typically referred to as Lateral Flow Immunoassays (LFIAs).

LFAs can be used for the detection of large complex analytes using a sandwich assay format or for the detection of small molecules or haptens using a competitive format. In a sandwich assay, typically a strip is assembled with a series of absorbent pad materials that direct the flow of sample and assay reagents across a series of discrete zones during which the target analyte is tagged (i.e. labelled) and subsequently captured and detected. The specimen is initially applied to an absorbent sample pad of the strip, which acts as a filter and a reservoir for the sample. Fluid is drawn, from the sample pad, through a conjugate release pad of the strip, where one or more target analytes in the sample are labelled by interacting with colorimetric, fluorescent, magnetic or radioactive reporter molecules. To effect the labelling, the reporter molecules are coupled to an analyte-specific ligand (usually an antibody), which rapidly forms complexes with respective target analytes to form labelled complexes. The sample including labelled complexes is drawn from the conjugate release pad to a test zone of the strip where one or more complementary ligands are immobilised onto the strip, at one or more test lines, to bind to the labelled complexes. Remaining sample is transferred from the test zone to a highly absorbent sink pad. The presence of any labelled complexes at the one or more test zones provides a measurable indication of the presence of the one or more target analytes in the sample. The test may be interpreted by the naked eye, for example, whereby the presence of one or more ‘visible’ test lines provides a qualitative indication of the presence of one or more target analytes.

The actuation of the fluid sample through a LFA does not normally require the input of energy, with migration of the sample being driven by capillary forces at the wet-dry interface (i.e. the leading edge of the liquid front) until it saturates the assay material (e.g., the nitrocellulose membrane) and is then drawn into the highly absorbent sink pad by wicking forces.

LFAs have traditionally suffered from a number of performance limitations, leading to limited analytical and clinical sensitivity, poor test-to-test reproducibility that largely limits its capacity to providing qualitative or binary measurements, and a reliance on visual interpretation due to challenges in the integration with affordable on-board electronics and built in quality control functions.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

According to one aspect of the present disclosure there is provided a method of performing a lateral flow test for making a determination about at least a first analyte of interest in a sample from a body, the method comprising:

applying a sample to a receiving portion of a lateral flow device such that the sample flows from the receiving portion to at least a first test zone and a second test zone of the lateral flow device,

monitoring levels of first and second signals at the first and second test zones over an assay period wherein, if the first analyte of interest is present in the sample, the first analyte is labelled and wherein the presence of labelled first analyte in the sample causes the level of one of the first and second signals to increase during the assay period; and

monitoring a change between the first and second signal levels over a period of time during the assay period.

According to another aspect of the present disclosure there is provided a lateral flow assay for making a determination about at least a first analyte of interest in a sample from a body comprising:
a lateral flow device, comprising:
   a receiving portion and at least first and second test zones, the receiving portion being configured to receive a sample such that the sample flows from the receiving portion to the first and second test zones, and
a reader configured to:
   monitor levels of first and second signals at the first and second test zones over an assay period wherein, if the first analyte of interest is present in the sample, the first analyte is labelled and wherein the presence of labelled first analyte in the sample causes the level of one of the first and second signals to increase during the assay period; and
   monitor a change between the first and second signal levels over a period of time during the assay period.

In some embodiments, the first and second signal levels may be adjusted to enable a more accurate comparison of the first and second signal levels during the monitoring of a change between the first and second signal levels. The adjustment of the first and second signal levels may include calibration and/or normalisation of the first and second signal levels, for example.

For example, the method and/or reader of the test device may:

identify a baseline level of the first and second signals that is prior to an initial time point at which a front of the sample arrives at the first and/or second test zone from the receiving portion, and

subtract the baseline level from the first and second signal levels to obtain calibrated first and second signal levels.

As another example, the method and/or reader of the test device may normalise the first and second signal levels, e.g., the calibrated first and second signal levels.

Thus, the monitoring of a change between the first and second signal levels during the assay period may comprising monitoring a change between the first and second signal levels as calibrated and/or normalised.

In some embodiments, labelling of the analyte(s) of interest may occur separately to the lateral flow process. The labelling may occur upstream of the lateral flow process, e.g., as part of an incubation process of otherwise. The sample may be prepared in a solute form. Any labelled complexes may be distributed relatively uniformly throughout the sample. A relatively homogenous labelled sample may therefore be received at the first and second test zones. This may provide, during the assay period, a substantially linear increase of the signal level at one of the test zones and a substantially constant signal level at the other one of the test zones, for example.

The monitoring of the change between the first and second signal levels may comprise monitoring a change in the difference between the first and second signal levels over the period of time. Differences between the signal levels may be calculated by subtracting one of the first and second signal levels from the other of the first and second levels, or by determining a ratio of the first and second signal levels. Differences between the signal levels may be calculated at one time point only, e.g. a single end point of testing (e.g. at the end of the assay period), or for different time points, e.g. at two or more time points during the assay period. The difference between the first and second signal levels at any time point may provide a delta value (A) or ratio value (R). The monitoring of the change between the first and second signal levels may comprise monitoring a change (e.g. an evolution) of the delta value (A) or ratio value (R) over the period of time during the assay period. The change of the delta or ratio value may be quantified in some embodiments.

In some embodiments, for example, the monitoring of the change between the first and second signal levels over a period of time during the assay period comprises at least:

comparing the first and second signal levels at a first time point to obtain a signal level difference (Δi) or a ratio value (Ri) at the first time point,

comparing the first and second signal levels at a second time point to obtain a signal level difference (Δf) or a ratio value (Rf) at the second time point, and

comparing the signal level difference (Δi) at the first time point with the signal level difference (Δf) at the second time point or comparing the ratio value (Ri) at the first time point with the ratio value (Rf) at the second time point.

The comparing of the signal level differences may comprise subtracting one of the signal level differences or one of the ratio values from the other, or obtaining a ratio of the signal level differences or ratio values.

The comparing of the signal level differences may provide for a quantification of a change in the delta value. The quantification may be provided as one or more test values, also referenced herein as an “S” values. Generally the test or S values will provide an indication of a degree of divergence between the first and second signal levels over certain time periods. In the above example, the S value may be calculated as follows: S=Δi−Δf or S=Δi/Δf or S=Ri−Rf or S=Ri/Rf. Alternatively, e.g. if the first and second signal levels are normalised at an earlier time point, an S value may be based on a signal level difference (Δ) or ratio value (R) for a single subsequent time point only, such as at a single intermediate time point (tᵢ) or single end time point (t_{end}) of testing. For example, the S value may be calculated as follows: S(tᵢ)=Δᵢ, or S(t_{end}), =Δ_{end}, or S(tᵢ)=Rᵢ, or S(t_{end})=R_{end}.

Alternative approaches to quantifying the change between the first and second signal levels may be carried out, however, e.g., to obtain a test value or otherwise. For example, gradients of lines indicative of the progression of the first and second signal levels lines may be calculated. A change in relative gradient between the first and second signal level lines may be calculated.

The monitoring of the change between the first and second signal levels may be used in the method and/or assay to make a determination about a medical condition. The determination about the medical condition may be based on whether or not the change is above or below a threshold change, for example. Where a test value is calculated, which test value may indicate a divergence between the first and second signal levels over a time period, the determination about the medical condition may be based on whether or not the test value is above or below a threshold value. In one embodiment a ‘positive’ test (i.e. presence of the medical condition) is identified if the test value at a test end point (t_{end}) exceeds a threshold value. For example, where an S value is obtained, a positive test may be identified if S(t_{end})>Sₘₐₓ. Additionally or alternatively, in one embodiment, a positive test is identified, even if the test value does not exceed the threshold value at the test end point (t_{end}), if a divergence between the first and second signal levels up to the test end point (t_{end}) is such that it can be predicted that the test value would exceed the threshold value at some time point in future, e.g., by regression analysis. For example, for successive time periods (t₁, t₂, t₃ . . . ) up to the test end point (t_{end}) a positive test may be identified if test values are continuously increasing, e.g. S(t₁)<S(t₂)<S(t₃) . . . . Thus, the method and/or assay may provide for forecasting of an end result, enabling a medical condition to be identified even if the level of a target analyte in the sample is relatively low.

Additionally or alternatively, the monitoring of the change between the first and second signal levels may be used to make a quantitative determination about a level (e.g. a concentration) of the first analyte in the sample and/or a human or animal body providing the sample. The change may be compared to a look-up table, one or more pre-determined signal curves or otherwise to make the quantitative determination. Where a test value is calculated, the quantitative determination about a level of the first analyte in the sample may be based on a look-up table in which test values are correlated with first analyte levels.

In some embodiments, monitoring a change between the first and second signal levels may comprise comparing the first and second signal levels at more than two time points, e.g., three of more time points. As an example, when at least three time points are used, the monitoring of the change may additionally comprise:

comparing the first and second signal levels at the third time point to obtain a signal level difference (Δg) or a ratio value (Rg) at the third time point,

comparing the signal level difference (Δg) at the third time point with the signal level difference (Δi, Δf) at the first and/or second time point or comparing the ratio value (Rg) at the third time point with the ratio value (Ri, Rf) at the first and/or second time point.

The use of at least a third time point for comparison may provide a further quantification of a change in the delta value. The quantification may provide a further test value. Where multiple test values are obtained, they may be averaged to arrive at a final test value.

The monitoring of a change between the first and second signal levels may be carried out over a time period that is sufficiently long to ensure that, if labelled first analyte is present in the sample, one of the first and second signal levels is seen to increase in a consistent manner in comparison to the other of the first and second signal levels.

The method and/or assay may be configured to wait for a predetermined period of time before monitoring a change between the first and second signal levels, e.g. after an initial time point at which a front of the sample arrives at the first and/or second test zone from the receiving portion.

In some embodiments, where signal level differences are compared at least at a first time point, the first time point may be at least 10 seconds, at least 20 seconds, at least 30 seconds, at least 1 minute, at least 2 minutes, at least 3 minutes, at least 4 minutes, at least 5 minutes, at least 6 minutes, at least 8 minutes or at least 10 minutes after the initial time point.

In some embodiments, where signal level differences are also compared at least at a second time point, after the first time point, the second time point may be at least 1 minute, at least 2 minutes, at least 3 minutes, at least 4 minutes, at least 5 minutes, at least 6 minutes, at least 8 minutes or at least 10 minutes after the initial time point.

The second time point may be at least 1 minute, at least 2 minutes, at least 3 minutes, at least 4 minutes, at least 5 minutes or at least 6 minutes after the first time point.

In general, it will be understood that references herein to comparing of signal levels at one or more time points are intended to indicate a comparison of the signals as they existed at those time points (optionally subject to, for example, time-shifting of signals to account for temporal lag, as discussed below). The comparison may be carried out substantially in real time or a later time, e.g., after signal data sets have been acquired for an entire testing period.

As indicated, the monitoring of the change between the first and second signal levels can be based on calibrated and/or normalised first and second signal levels. While normalisation and calibration may be preferred, in alternative aspects one or both of the calibration and normalisation steps may be omitted, e.g. if the approach is to be used for a qualitative, rather than a quantitative, determination about the first analyte or associate medical condition, and/or for a cruder determination about the first analyte or associated medical condition.

Where calibration is used, as indicated above, a baseline level of the first and second signals, that is prior to an initial time point at which a front of the sample arrives at the first and/or second test zone, is calculated. The baseline level is subtracted from the first and second signal levels after the initial time point. The baseline level may be indicative of a “dry read” for the first and second signals at the first and second test zones. The baseline level may be indicative of the signals level at the first and second test zones that does not result from the presence of the sample, including any labelled analyte, at that test zone. By subtracting the baseline level from the first and second signal levels, background noise may be removed. Preferably, first and second baseline levels are calculated and subtracted from the first and second signal levels respectively. Nevertheless, it is conceived that a single baseline level may be calculated only and subtracted from the first and second signal levels.

Where normalisation is used, the first and second signal levels may be normalised, for example, based on their signal levels when the sample arrives at the first and second test zones and provides an initial signal level peak. Alternatively, the first and second signal levels may be normalised, for example, based on their signal levels after a signal level peak, such as at an early time point after the peak. The normalisation approach based on signal levels after the initial peak signal may be preferred if it is hard to discern sufficiently precise peak signal levels due to the resolution of the signal data and possible rounding of the peak signal profiles. The initial peak for each of the first and second signals may be substantially at an initial time point at which a front of the sample arrives at the first and/or second test zone from the receiving portion, or very soon after the initial time point. The normalisation may be such that the levels of the initial peaks for the first and initial signal levels, or later values for the first and second signal levels, are matched.

In some embodiments, one of the first and second test zones may be further from the receiving portion than the other of the first and second test zones. Accordingly, the sample may take longer to reach one of the first and second test zones in comparison to the other of the first and second test zones. In some embodiments, the first and signals may therefore be time shifted relative to each other, e.g. by the reader, prior to determining changes between the first and second signal levels after the initial time point. The first and second signals may be time-shifted to compensate for delays in the sample reaching the test zone that is furthest from the receiving portion. Thus, the monitoring of the change between the first and second signal levels after the initial time point may be based on the first and second signals as time-shifted relative to each other.

The time-shifting may compensate for an increasing delay in the sample reaching the test zone that is furthest from the receiving portion, in comparison to the test zone that is closest to the receiving portion. The time-shifting may be based on a lag-coefficient that accounts for the increasing delay. The lag-coefficient may therefore provide for dynamic time-shifting of the first and second signals during the assay period. Alternatively, however, a fixed time-shift of the first and second signals may be employed.

In any one or more of the above aspects, the method or assay may also be for making a determination about a second analyte of interest in a sample from the body. If the second analyte of interest is present in the sample, the second analyte may be labelled in the sample. The monitoring of the levels of first and second signals at the first and second test zones over an assay period may recognise that, if labelled first analyte is present in the sample, the level of one of the first and second signals may increase during the assay period and, if labelled second analyte is present in the sample, the level of the other one of the first and second signals may increase during the assay period. The presence of the second analyte of interest in the sample may be mutually exclusive of the presence of the first analyte of interest in the sample. The first analyte of interest may be an Influenza A analyte and second analyte of interest may an Influenza B analyte, or vice versa, for example.

The method and/or assay of the present disclosure may employ various conventional lateral flow techniques, which may rely on the forming of a sandwich assay, for example. Prior to being received at the first and second test zones, the sample may be combined with a first mobilisable capture reagent that is able to bind specifically to the first analyte of interest, if present in the sample, to form a plurality of first labelled complexes. One of the first and second test zones may comprise a first immobilised capture reagent being able to bind specifically to the first labelled complexes to immobilize the first labelled complexes. On the other hand, the other of the first and second test zones may be configured so that it does not immobilize or has a reduced ability to immobilize a plurality of the first labelled complexes. Thus, when the analyte of interest is present in the sample, the labelled complexes may accumulate at one of the first and second test zones, and not the other.

As indicated above, in some embodiments, labelling of the analyte(s) of interest may occur separately to the lateral flow process. The labelling may occur upstream of the lateral flow process, e.g., as part of an incubation process of otherwise. The sample may be prepared in a solute form, such that any labelled complexes are distributed relatively uniformly throughout the sample.

In more detail, the sample may be incubated with at least a first mobilisable capture reagent comprising detectable labels, wherein the first mobilisable capture reagent is able to bind specifically to the first analyte of interest, if present in the sample, to form a plurality of first labelled complexes. After the incubating, the sample may be applied to a receiving portion of the lateral flow device such that the sample, including any labelled complexes, flows from the receiving portion to at least the first and second test zones of the lateral flow device.

In one aspect of the present disclosure there is provided apparatus comprising the lateral flow assay and an incubation vessel for incubating the sample.

In any one or more of the above aspects, after the initial time point at which a front of the sample arrives at the first and/or second test zone from the receiving portion, if first labelled complexes are present in the sample, the level of one of the first and second signals may increase in a substantially linear manner. This may be due to a homogeneity of the first labelled complexes in the sample, particularly if incubation has been carried out. The first labelled complexes may be progressively immobilized at the respective test zone.

On the other hand, after the initial time point, the level of the other one of the first and second signals may remain substantially the same during the assay period, while being a non-zero level. This may be due to a homogeneity of the first labelled complexes in the sample after incubation, which complexes are moving through the respective test zone without being immobilized, yet providing for a continuous signal.

By providing for a substantially linear increase of the signal level at one of the test zones, while the signal level remains substantially the same non-zero level at the other of the test zones, an assay with increased sensitivity and/or with the ability provide earlier detection, may be achieved. The linearity of the increasing signal level may allow extrapolation of data for a period longer than the assay period, for example, allowing forecasting of test results, for example. Moreover, the level that remains substantially the same may provide for a base signal level against which the other signal level can be accurately and reliably compared.

As indicated, the incubating of the sample may form a substantially homogeneous mixture of labelled complexes. The incubating may be carried out for a period of at least 30 seconds, at least 1 minute, at least 2 minutes, at least 5 minutes, at least 7 minutes, at least 10 minutes, at least 15 minutes, at least 20 minutes, at least 25 minutes or at least 30 minutes.

The incubating may comprise mixing the sample with a buffer solution. The incubating may be carried out by depositing the sample into the interior of a vessel, the interior of the vessel being separate from the lateral flow device. Prior to the depositing of the sample into the interior of the vessel, the at least a first mobilisable capture reagent may located on an inner surface of the vessel. Additionally or alternatively, the at least a first mobilisable capture reagent may be coated on or otherwise located in a separate item, such as a pad, and may be located in the vessel prior to, after or at the same time as the deposition of the sample in the vessel.

The labels may be fluorescent labels. The fluorescent labels may comprise one or more quantum dots. Nevertheless, gold nanoparticles or a variety of other labels such as coloured latex beads, magnetic particles, carbon nanoparticles, selenium nanoparticles, silver nanoparticles, up converting phosphors, organic fluorophores, textile dyes, enzymes, liposomes and others may be used.

The first and second signals may be produced by monitoring one or more physical parameters at the first and second test zones using one or more detectors. Where the labels are fluorescent labels, dyes or otherwise, the first and second signals may be produced by detecting changing intensities of light at the first and second test zones. The levels of the first and second signals may be proportional or inversely proportional to the levels of light detected at the first and second test zones, for example. As another example, where the labels are magnetic particles, the first and second signals may be produced by detecting changing magnetic field strengths at the first and second test zones. The levels of the first and second signals may be proportional or inversely proportional to the magnetic field strength detected at the first and second test zones, for example.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the present disclosure are now described by way of example with reference to the accompanying drawings, in which:

FIG. 1 shows an oblique view of a lateral flow assay according to an embodiment of the present disclosure;

FIG. 2 is a flowchart illustrating features of a method for making a determination about at least a first analyte of interest in a sample, according to an embodiment of the present disclosure;

FIG. 3a shows an oblique view of an incubation vessel receiving a sample in accordance with an embodiment of the present disclosure;

FIG. 3b shows an oblique view of the incubation vessel of FIG. 3a with the sample being incubated for a period of time;

FIG. 3c shows application of the sample after incubation to the lateral flow assay of FIG. 1;

FIG. 4 is a graph of normalised signal strength of first and second signals detected at first and second test zones of a lateral flow assay, employing a peak clearance testing approach;

FIG. 5a is a graph of signal strength of first and second signals detected at first and second test zones of a lateral flow device, following incubation of the sample prior to application of the sample to the device (the units of signal strength are in Hz based on a light intensity to frequency conversion by a photodetector);

FIG. 5b is a graph corresponding to the graph of FIG. 5a, but with the first and second signals normalised and time-shifted in accordance with an embodiment of the present disclosure;

FIG. 6 is a flowchart illustrating features of a method for making a determination about at least a first analyte of interest in a sample, according to an embodiment of the present disclosure;

FIG. 7 is another flowchart illustrating features of a method for making a determination about at least a first analyte of interest in a sample, according to an embodiment of the present disclosure;

FIG. 8 is a flowchart illustrating features of a method for making a determination about at least a first analyte of interest and a second analyte of interest in a sample, according to an embodiment of the present disclosure;

FIG. 9 is a graph illustrating a correlation of S value with Flu B analyte concentration calculated obtained using a method according to an embodiment of the present disclosure;

FIG. 10 is a graph illustrating a correlation of S value with analyte purified CRP antigen concentration obtained using a method according to an embodiment of the present disclosure;

FIGS. 11a and 11b are graphs illustrating the performance of an accretion method vs a conventional peak clearance method where the target analyte is an Influenza A antigen and an Influenza B antigen, respectively;

FIGS. 12a and 12b are graphs of signal strength of first and second signals detected at first and second test zones of a lateral flow device before and after calibration/normalisation, respectively;

FIG. 13 is a graph of signal strength of first and second signals detected at first and second test zones of a lateral flow device, which have been normalised at an early time point after a signal peak;

FIG. 14 is a graph of signal strength of first and second signals detected at first and second test zones of a lateral flow device, which have been normalised and which are illustrative of a weak positive test; and

FIG. 15 is a flow chart indicative of decision making by a reader to determine a positive test result including through forecasting.

### DETAILED DESCRIPTION OF EMBODIMENTS

Embodiments of an apparatus and a method for performing a lateral flow test, for making a determination about at least a first analyte of interest in a sample from a body, are now described. The apparatus and method may provide for a quantitative or semi-quantitative determination about at least the first analyte of interest to be made. In some embodiments, the determination about at least the first analyte of interest may provide or lead to a determination about a medical condition of a human or animal body from which the sample was received.

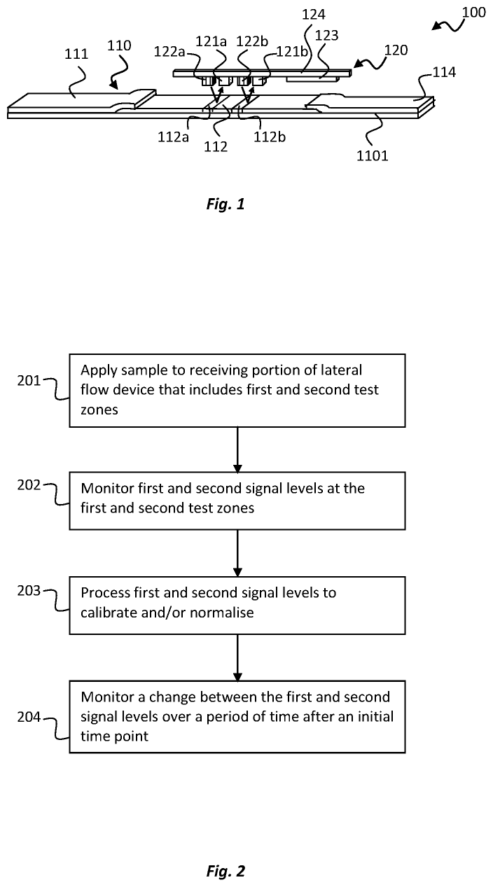

FIG. 1 provides an illustration of components of an assay 100 according to an embodiment of the present disclosure and FIG. 2 provides a flowchart 200 of features carried out in a method according to an embodiment the present disclosure that can use the assay.

As shown in FIG. 1, a lateral flow device 110 of the assay 100 in this embodiment has a series of absorbent pad materials, located on a waterproof backing layer 1101, the pad materials directing the flow of sample through the device 110 (generally in a left to right direction as depicted) by virtue of capillary action. The absorbent pad materials may be formed of any material which permits flow of a liquid sample therethrough by capillary action and which is known to be suitable for use in lateral flow devices. Such materials have been widely used in commercially-available diagnostic tests and will be known to a person skilled in the art.

Referring also to FIG. 2, at 201, the sample is applied to a receiving portion 111 of the lateral flow device 110 such that the sample flows from the receiving portion 111 to at least a first test zone 112a and a second test zone 112b of the lateral flow device 110. The later flow device 110 includes a fluid sink 114, which may act to draw the sample through or along the absorbent pad material in the device 110.

At 202, levels of first and second signals at the first and second test zones 112a, 112b over an assay period are monitored. If first analyte of interest is present in the sample, the first analyte is labelled in the sample, prior to reaching the first and second test zones. If labelled first analyte is present in the sample, the level of at least one of the first and second signals increases during the assay period.

Referring again to FIG. 1, in this embodiment a reader 120 is provided that monitors the first and second signals at the first and second test zones 112a, 112b to determine first and second signal levels over a period of time. The reader 120, in combination with the lateral flow device 110, provides the lateral flow assay 100. The reader 120 comprises electrical components including first and second photodetectors 121a, 121b, mounted on a printed circuit board (PCB) 124, along with a processor 123. The first and second photodetectors 121a, 121b detect an intensity of light at the first and second test zones 112a, 112b, respectively. Light may be reflected, absorbed and/or emitted at the first and second test zones 112a, 112b to different degrees, dependent on the number and type of detectable labels present at the first and second test zones 112a, 112b, for example. The levels of the first and second signals may calculated as a value that is proportional or inversely proportional to the level of light detected at the first and second test zones 112a, 112b, for example.

At 203, processing of the signals/signal levels is carried out, e.g., by the reader 120 or more specifically the processor 123 of the reader. For example, in the processing, identification is made of a baseline level of the first and second signals that is prior to an initial time point at which a front of the sample arrives at the first and/or second test zone 112a, 112b from the receiving portion 111. The baseline level can be subtracted from the first and second signal levels to obtain calibrated first and second signal levels after the initial time point. The baseline level may be indicative of a “dry read” for the first and second signals at the first and second test zones 112a, 112b. The baseline level may be indicative of the signals level at the first and second test zones 112a, 112b that does not result from the presence of the sample, including any labelled analyte, at that test zone. By subtracting the baseline level from the first and second signal levels, background noise may be removed. Preferably, first and second baseline levels are calculated and subtracted from the first and second signal levels respectively. Nevertheless, it is conceived that a single baseline level may be calculated only and subtracted from the first and second signal levels.

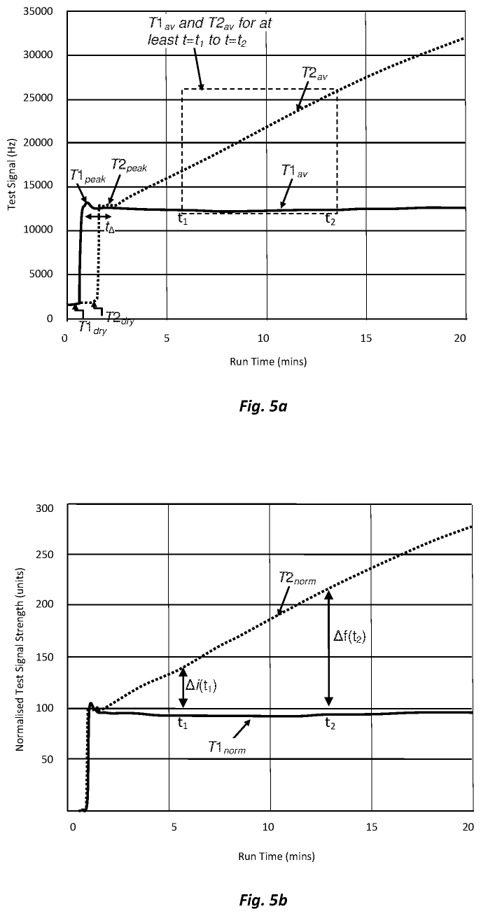

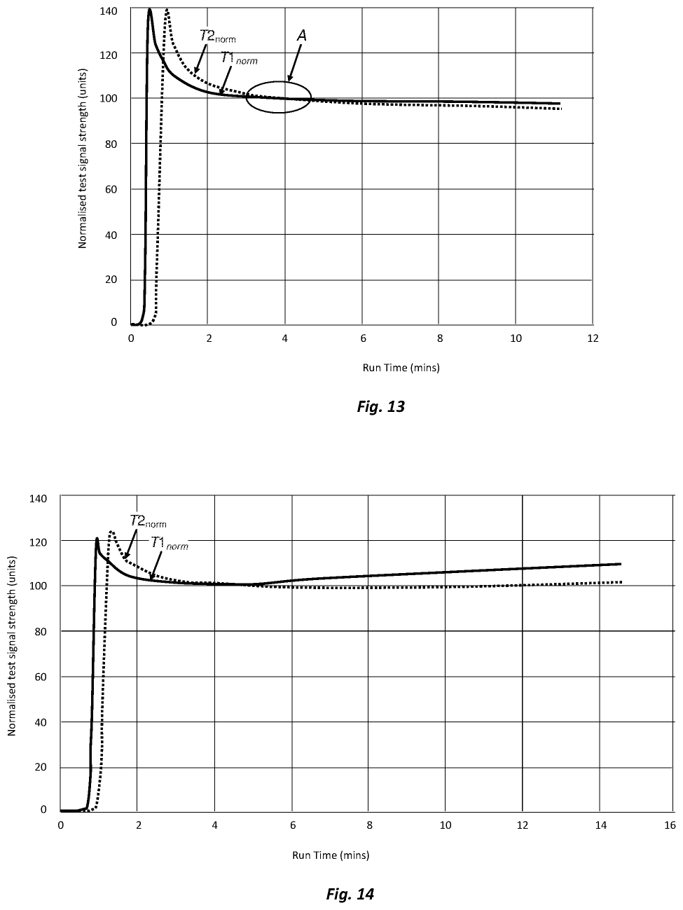

In the processing, the first and second signal levels can also be normalised. The first and second signal levels can be normalised, for example, based on their signal levels when the sample arrives at the first and second test zones and provides an initial signal level peak, or after the initial signal level peak when the sample arrives at the first and second test zones. The initial peak for each of the first and second signals may be substantially at the initial time point or very soon after the initial time point. The normalisation may be such that the levels of the initial peaks for the first and second signals are matched. One example of such normalisation is discussed further below, with reference to the graphs of FIGS. 5a and 5b. Alternatively, the normalisation may be such that the levels for the first and second signals are matched at a time after the initial peaks, e.g. a relatively early time such as between 30 seconds and 5 minutes after the initial peaks. An example of this is illustrated in FIG. 13, where the first and second signals have been normalised at a time period identified by region A.

At 204, a change between the processed first and second signal levels over a period of time during the assay period, after the initial time point, is monitored, e.g., by the processor 123 of the reader 120. The monitoring of the change between the first and second signal levels may comprise monitoring a change in the difference between the first and second signal levels over the period of time. Differences between the signal levels may be calculated by subtracting one of the first and second signal levels from the other of the first and second levels, or by determining a ratio of the first and second signal levels. Differences between the signal levels may be calculated at one time point only, e.g. a single end point of testing (e.g. at the end of the assay period), or for different time points, e.g. at two or more time points during the assay period. The difference between the first and second signal levels at any time point may provide a delta (A) value or a ratio value (R). The monitoring of the change between the first and second signal levels may comprise monitoring a change (e.g. an evolution) of the delta value (A) or ratio value (R) over the period of time during the assay period. The change of the delta or ratio value may be quantified in some embodiments. Nevertheless, alternative approaches to quantifying the change between the first and second signal levels may be carried out, however, to obtain a test value or otherwise. For example, gradients of lines indicative of the progression of the first and second signal levels lines may be calculated. A change in relative gradient between the first and second signal level lines may be calculated. One example of how the change between the processed first and second signal levels is monitored over a period of time is again discussed further below, with reference to the graphs of FIGS. 5a and 5b.

In this embodiment, prior to application to the receiving portion 111 of the lateral flow device 110, the sample is incubated to label any first analyte of interest present in the sample. Labelling can be carried out by incubating the sample with at least a first mobilisable capture reagent comprising detectable labels. During the incubation, the first mobilisable capture reagent may bind specifically to the first analyte of interest, if present in the sample, to form a plurality of first labelled complexes.

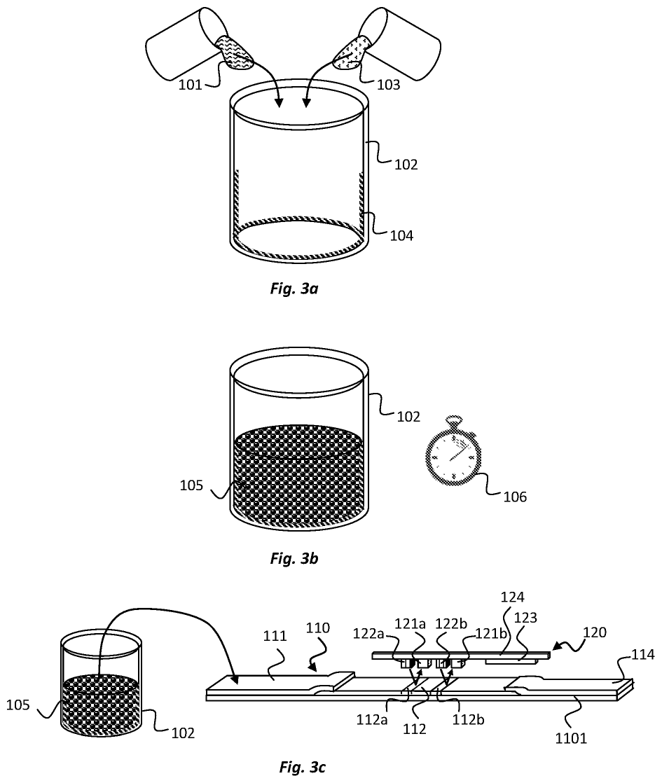

As illustrated in FIG. 3a, in the incubation process according to one embodiment, a sample 101 is deposited into an interior of a vessel 102. To increase the fluidity of the sample 101, a buffer solution 103 can also be deposited in the vessel 102. The deposition of the buffer solution 103 can be prior to, after, or at the same time as, the deposition of the sample 101 in the vessel 102, such that the buffer solution 103 mixes with the sample 101. In this embodiment, at least a first mobilisable capture reagent 104 is coated on an inner surface of the interior of the vessel 103, prior to receipt of the sample. In alternative embodiments, the first mobilisable capture reagent 104 may be coated on or otherwise located in a separate item, such as a pad, and may be located in the vessel prior, after or at the same time as the deposition of the sample in the vessel.

When deposited in the vessel 102, the sample 101, the buffer solution 103 if present, and the first mobilisable capture reagent 104, can form a sample mixture 105 as represented generally in FIG. 3b. In the sample mixture 105, binding of the first mobilisable capture reagent to the first analyte of interest, if present in the sample, takes place.

As represented by a timer 106 in FIG. 3b, the incubation can take place for a certain period of time, such as a period of time that is sufficient to cause a homogeneous mixture of first labelled complexes to form in the mixture. For example, incubation can be carried out for at least 30 seconds, at least 1 minute, at least 2 minutes, at least 5 minutes, at least 7 minutes, at least 10 minutes, at least 15 minutes, at least 20 minutes, at least 25 minutes or at least 30 minutes or otherwise. After incubation, the sample can be applied to the receiving portion of the test device, generally as represented in FIG. 3c.

While the vessel in FIGS. 3a to 3c is a separate item from the lateral flow assay/lateral flow device 110, in alternative embodiments it may be combined with the lateral flow device. For example, a vessel may be attached to the lateral flow device. While attached to the lateral flow device, it may be transitionable from a first state, where its contents are fluidly isolated from the lateral flow device, to a second state, where its contents are fluidly connected to the lateral flow device. Incubation may occur while the vessel is in the first state and then, when transitioned to the second state, the contents (e.g. the sample mixture) may automatically transfer onto the receiving portion of the lateral flow device. In embodiments of the present disclosure, the vessel may take any form suitable for holding a liquid. For example, it may be configured as a cup, a tube, an absorbent pad, a dropper or otherwise.

By incubating the sample mixture prior to application to the receiving portion 111 of the lateral flow device 110, the lateral flow device 110 may be absent of any conjugate release pad for labelling the sample. The incubation of the sample prior to the lateral flow process can be advantageous by providing for homogeneity in the distribution of labels in the sample, prior to the sample reaching the first and second test zones, as discussed in more detail below. Nevertheless, in alternative embodiments, different approaches to sample preparation, prior to the sample reaching the first and second test zones, may be employed, including through use of a conjugate release pad as part of the test device or otherwise.

In this embodiment, one of the first and second test zones 112a, 112b is configured to immobilize a plurality of the first labelled complexes, and the other of the first and second test zones 112a, 112b is configured so that it does not immobilize (or at least has a reduced ability to immobilize) the first labelled complexes. When the sample travels to each of the first and second test zones 112a, 112b, any first labelled complexes present in the sample provides for an increase in the first and second signals at the first and second test zones 112a, 112b. The first and second signals are generally indicative of the levels of the first labelled complexes at the first and second test zones, respectively, at any instant in time.

While either one of the first and second test zones 112a, 112 can be configured to immobilize a plurality of the first labelled complexes, in this embodiment, the second test zone 112b is configured to immobilize a plurality of the first labelled complexes. To immobilize the plurality of the first labelled complexes, the second test zone 112b comprises a first immobilised capture reagent being able to bind specifically to the first labelled complexes. In this embodiment, the first test zone 112a does not immobilize any first labelled complexes as it includes little or no capture reagents that are able to bind specifically to the first labelled complexes. In fact, in this embodiment, the first test zone 112a is substantially indistinct from immediately adjacent portions of the test device 110.

In the present embodiment, the labels are fluorescent labels, such as fluorescent labels comprising one or more fluorescent quantum dots. The fluorescent labels are configured to fluoresce at one or more specific wavelengths detectable by the photodetectors 121a, 121b. The fluorescent labels are caused to fluoresce, and therefore emit an emission light signal, upon excitation by an incident excitation light signal. In this embodiment, the excitation light is provided by first and second emission light sources such as first and second LEDs, 122a, 122b. By using fluorescent labels in the present embodiment, the levels of the first and second signals may be directly proportional to the levels of emission light detected at the first and second test zones by the photodetectors. Waveguides and/or optical filters may be located between the test zones 112a, 112b and the photodetectors and/or LEDs. In alternative embodiments, a single photodetector may be used to monitor emission light at the first and second test zones, e.g. to obtain first and second signals as a time-multiplexed signal.

The reader 120 of this or any other embodiment may be at least partly integrated with the lateral flow device 110, e.g. by being located, in combination with at least the test portion 112 of the lateral flow device 110, in a common housing. The housing may minimise any ambient light that may otherwise be detected by the photodetectors. Alternatively, all or part of the reader may be located in a separate device that is connectable to the lateral flow device. The separate device may be an electronic base unit. The electronic base unit may provide power to components of the reader whether the components of the reader are located in the base unit or elsewhere. The electronic base unit may comprise a port to receive the lateral flow device. The results of testing may be presented on a display that forms part of the reader and/or separate device.

By employing fluorescent labels, sensitivity gains may be achieved over more commonly deployed labels in assays, such as gold nanoparticles (colloidal gold). Nevertheless, gold nanoparticles or a variety of other labels such as coloured latex beads, magnetic particles, carbon nanoparticles, selenium nanoparticles, silver nanoparticles, up converting phosphors, organic fluorophores, textile dyes, enzymes, liposomes and others may also be used in embodiments of the present disclosure.

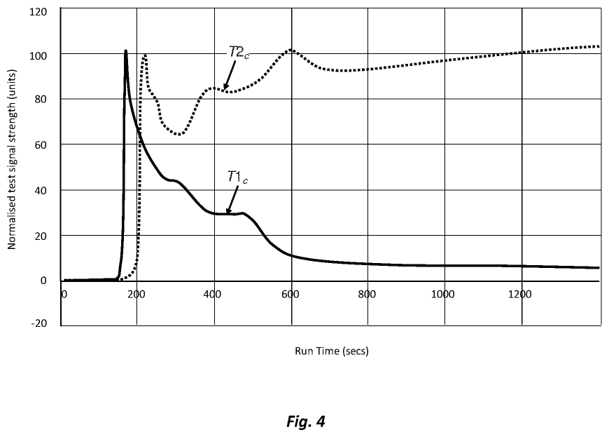

Example behaviours of first and second signal levels, processing of the first and second signal levels, and monitoring of changes between the first and second signal levels is now described with reference to the graphs of FIGS. 4, 5a and 5b.

FIG. 4 provides a graph of signal strength for normalised first and second signals detected at first and second test zones of a lateral flow assay. In this example, there has been no incubating of a sample, containing the first analyte of interest, prior to application to the lateral flow device. Instead, a conjugate release pad, including a dessicated first mobilisable capture reagent, comprising fluorescent labels, is provided as part of the lateral flow device, with binding of the mobilisable capture reagent to the first analyte of interest, to form first labelled complexes, taking place only as the sample washes through the conjugate release pad.

In this “peak clearance” method, rapid rehydration and release of the desiccated reagent, following the deposition of the sample, creates a high concentration of the labelled complexes in the leading edge of the fluid front. As seen in FIG. 4, which shows the strength of the first and second signals T1_{c}, T2_{c}, detected at the first and second test zones respectively, the highly concentrated leading edge is represented by a large and sharp signal peak shortly after the initiation of the monitoring and of the lateral flow process. The peak occurs both for the second signal T2_{c} detected at the second test zone, where immobilization of the first labelled complexes occurs, and for the first signal T1_{c} detected at the first test zone where no immobilization occurs and where the first labelled complexes simply wash through the first zone. At the second test zone, after the highly concentrated leading edge passes through, the level of the second signal T2_{c} drops and then increases as the assay develops and more labelled complexes are immobilized. At the first test zone, the level of the first signal T1_{c} drops as the labelled complexes clear, approaching a near original baseline or initial ‘dry’ signal level.

As can be seen in FIG. 4, even in this example of a strong ‘positive’ test, the changes in both the first and second levels over time are relatively uneven. Moreover, by dropping close to the initial dry signal level, the first signal provides a weaker signal against which the second signal can be consistently compared. Thus, while a “peak clearance” method, e.g. as represented in FIG. 4, may be employed in embodiments of the present disclosure, in some embodiments it may be preferable to incubate the signal prior to application to the lateral flow test device.

FIG. 5a provides a graph of signal strength for first and second signals T1, T2 detected at the first and second test zones of a lateral flow device according to an embodiment of the present disclosure in which the sample has been incubated prior to application to the lateral flow test device. FIG. 5a has been obtained on the basis of a nasal sample, the sample being analysed and testing positive for the Influenza virus. Prior to application to the lateral flow device, the sample, containing the first analyte of interest, has been incubated with a buffer solution and a mobilisable capture reagent including fluorescent labels for about 1 minute. As indicated, a fundamental difference between this approach, and the conventional peak clearance approach described above with reference to FIG. 4, is the relocation of an analyte-specific labelled capture reagent from a release pad on the test device itself to the upstream incubation vessel. This enables pre-mixing and pre-incubation of the capture reagent with the sample to form a sample mixture that has a homogeneous distribution of first labelled complexes, before application to the lateral flow device.

At the first test zone, no immobilization of labelled complexes takes place. However, as evident from FIG. 5a a consistent base signal, T1 is still present for the full duration of the testing. This is due to the homogeneity of the incubated sample mixture as it travels through the first test zone. At the second test zone, there is a gradual accumulation of the strength of the second signal T2, over the base signal T1, as first labelled complexes are immobilized at the second test zone. This “accretion” of the first labelled complexes is substantially linear.

Thus, in this “accretion method” example, according to the present disclosure, the first signal T1 can provide a base against which the second signal T2 can be more accurately compared. The comparison can be made at least at a period after the initial time point when the front of the sample arrives at the first and second time zones. In some embodiments, the comparison can be made at least at a first time point and at a second time point. By making a comparison at two different points in time, relative to at least the baseline signal, the degree of accretion of first labelled complexes at the second test zone can be more precisely monitored.

In general, FIG. 5a indicates that, following incubation of sample with the mobilised capture reagent, there is a substantially linear accretion of labelled complexes at one of the test zones (the second test zone in this example). The linearity of the results indicate that there is no need for complete clearance of the labelled complexes through the test zones to discriminate positives (including low positives) from background signal as the labelled complexes can interact consistently with the test zones for the whole duration of the test (a 20 minute duration in this example). The capillary force driving the fluidics progressively diminishes, thus providing longer time for the particles to interact at the test zone and generate signal. The linearity of the results allow derivation of a test value, e.g. an “S value” or a line gradient value, that can be used to quantitatively analyse the analyte of interest, as discussed in more detail below.

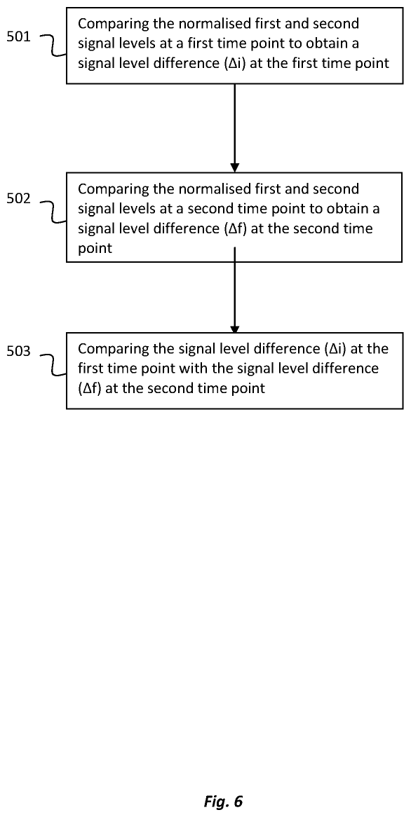

Referring to the flow chart of FIG. 6, in embodiments of the present disclosure, the following features can be carried out: at 501, comparing the first and second signal levels at a first time point to obtain a signal level difference (Δi) at the first time point, at 502, comparing the first and second signal levels at a second time point to obtain a signal level difference (Δf) at the second time point, and, at 503, comparing the signal level difference (Δi) at the first time point with the signal level difference (Δf) at the second time point. The comparing of the signal level differences may produce a test value, referred to herein as the “S value”, for example. A determination about a medical condition of a human or animal body from which the sample was received may be based on whether or not the test value or S value is above or below one or more threshold values. Nevertheless, test values or “S values” may be determined in other ways. For example, rather than comparing signal levels by subtraction to obtain delta values, ratios of the first and second signal levels can be obtained for different time points and the ratios may be compared. Moreover, the S value need not necessarily be based on signal level differences at multiple time points. For example, a signal level difference may be calculated at an end point of testing only.

In accordance with discussions above, prior to carrying out the comparing of the first and second signals, the first and second signals can be normalised to account for stray light and/or asymmetric efficiency at each test zone. Stray light can occur due to the excitation LEDs or ambient light ‘leaking’ to the photodetector(s), thus generating a constant background signal irrespectively of the presence of sample/fluorescent labels. Moreover, there may be a small misalignment of the optical components, tolerance stacks or deformation leading to asymmetric efficiency. In these respects, absolute measurement of fluorescent emissions from either of the test zones is not necessarily representative of the actual number of fluorescent labels immobilized or present at either one of the test zones. In the present disclosure, normalisation can be used to correct for imbalances between the first and second test zones. Prior to, or as part of the normalisation procedure, the first and second signal levels can be calibrated. In the calibration, raw measurements T1, T2 at the first and second test zones are corrected for their dry read measurements T1_{dry}, T2_{dry} that result from stray light or asymmetry. Thus, based on this correction, both the dry measurements can be reduced to zero. Moreover, in the normalisation procedure, signal levels, e.g. the peak signal levels T1ₚₑₐₖ, T2ₚₑₐₖ at the conjugate wavefront, as corrected based on the dry read measurements, are normalised to 1, 100 or another desirable number.

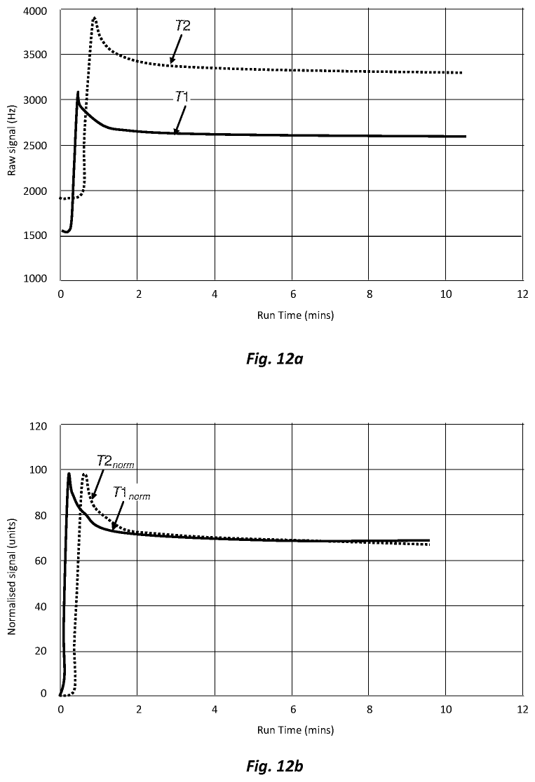

The importance of calibrating and normalising the first and second signal levels T1, T2 is further emphasised with reference to FIGS. 12a and 12b. FIG. 12a provides an example of first and second signal levels T1, T2 (for a negative sample in this instance) that have significantly different dry read measurements and therefore signal levels. FIG. 12b illustrates the calibrated and normalised dry read measurements.

The calibration and normalisation steps can enable more precise monitoring of the time evolution of the parameter delta (Δ) or ratio value (R), that indicates the difference in signal levels (strength) between T1 and T2. By measuring the parameter delta or ratio value at least at one time point after the initial time point, and sometimes at least at two or more time points, a correlation between the divergence in the post-peak phase to the accumulation of fluorescent labels at either test line, and thus the level of first the analyte present in the sample, can be made. It is recognised that the accumulation of labelled complexes at the test zones can be inferred by determining the parameter delta/ratio value at a single time point only, e.g. at an end time (t_{end}) of the assay (e.g. 6 minutes from the arrival of the conjugate wavefront). However, by monitoring the time-evolution of the delta/ratio value at least at first and second time points (e.g. by comparing it at 3 and 6 minutes, for example) advantages can be achieved. The comparison can help compensate for simultaneous drift of the fluorescence intensity at the test line (e.g. non-specific binding), for example. In addition, it can enable an expansion of the dynamic range of the assay (e.g. linear response over multiple decades of analyte concentration). Moreover, it may allow for forecasting of an end result on the basis of which a test result may be considered positive.

In general, the comparing of the first and second signals may occur for one or more time points at least 10 seconds, at least 20 seconds, at least 30 seconds, at least 1 minute, at least 2 minutes, at least 3 minutes, at least 4 minutes, at least 5 minutes, at least 6 minutes, at least 8 minutes or at least 10 minutes after the initial time point or otherwise. When comparisons are made at least at first and second time points, the first time point may be at least 10 seconds, at least 20 seconds, at least 30 seconds, at least 1 minute, at least 2 minutes, at least 3 minutes, at least 4 minutes, at least 5 minutes, at least 6 minutes, at least 8 minutes or at least 10 minutes after the initial time point or otherwise. Moreover, the second time point may be after the first time point and at least 20 seconds, at least 30 seconds, at least 1 minute, at least 2 minutes, at least 3 minutes, at least 4 minutes, at least 5 minutes, at least 6 minutes, at least 8 minutes or at least 10 minutes after the initial time point or otherwise. Further, the second time point may be at least 10 seconds, at least 20 seconds, at least 30 seconds, at least 1 minute, at least 2 minutes, at least 3 minutes, at least 4 minutes, at least 5 minutes or at least 6 minutes after the first time point or otherwise.

In general, it will be understood that references herein to reading or comparisons of signals for, or at, one or more time points after an initial time point, or at specific times after the initial time point (e.g. at 3 minutes, or 6 minutes), etc., are intended to indicate a reading or comparison of the signals as they existed at those time points (subject to time-shifting to account for temporal lag, as discussed below). The actual comparing may be carried out substantially in real time, or at a later time, e.g., after signal data sets have been acquired for an entire assay period.

The computation of the parameter delta or ratio value, in addition to accounting for stray light and asymmetric efficiency at the first and second test zones, can take into account the sample having to travel further onto the test strip before reaching one of the test zones in comparison to the other of the test zones. For example, in the device 110 as illustrated in FIG. 1, the sample will have to travel further to reach the second test zone 112b in comparison to the first test zone 112a. Accordingly, before computing the parameter delta or ratio value, the T1 and T2 signals can be aligned to compensate for a temporal lag t_{Δ} caused by the different positions of the first and second test zones. The temporal lag may increase over a period of time.

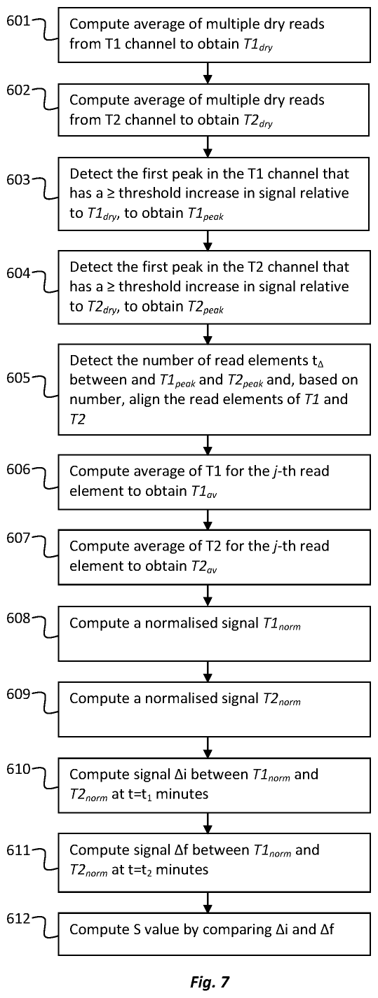

An example process for calibration and normalisation of the first and second signals T1, T2, the correction for temporal lag to, and the monitoring of the time-evolution of the parameter delta Δ, is now described in more detail with reference to the flowchart of FIG. 7. The process is based on the first and second signals being obtained by the reader, across respective first and second data channels, as an array of N data elements indicative of signal strength readings at multiple points in time, where the i-th data element is referenced for the two channels as T1(i) and T2(i), respectively. The data elements may be stored in a memory device that may be located in the reader and the process may be conducted by the processor of the reader.

At 601, an average of multiple dry readings for the T1 channel is computed to obtain T1_{dry}.

Similarly, at 602, an average of multiple dry readings for the T2 channel is computed to obtain T2_{dry}.

At 603, the first peak in the readings from the T1 channel is detected as a greater than threshold increase in the signal strength relative to T1_{dry}, e.g., a ≥20% increase, to obtain T1ₚₑₐₖ. Any secondary peaks, caused to a delay in particle release can be discarded.

Similarly, at 604, the first peak in the readings from the T2 channel is detected as a greater than threshold increase in the signal strength relative to T2_{dry}, e.g., a ≥20% increase, to obtain T2ₚₑₐₖ. Again, any secondary peaks, caused to a delay in particle release can be discarded.

Example graphical illustrations of T1_{dry}, T2_{dry}, T1ₚₑₐₖ, and T2ₚₑₐₖ are provided in FIG. 5a.

At 605, the number of read elements t_{Δ} between and T1ₚₑₐₖ and T2ₚₑₐₖ is detected and, based on this number, the read elements of T₁ and T₂ are aligned. This alignment accounts for a temporal lag between the T1 and T2 channels, caused by the differing positions of the first and second test zones on the test strip. Through use of a temporal lag co-efficient n, which can be specific to the material used in the test device and the viscosity of the sample, the alignment can provide for a dynamic alignment of the T1 and T2 read elements over the entire assay period. This is represented in Equation 1a below, which assumes that the second test zone is further from the sample receiving portion than the first test zone. The co-efficient n may be a number other than 1, such as approximately 2, for example.

T2(i)=T2(i−nt_{Δ}) Equation 1a

In alternative embodiments, however, a fixed time-shift of the T1 and T2 channels may be employed. This is represented in Equation 1b below, where N is an integer, and which again assumes that the second test zone is further from the sample receiving portion than the first test zone.

T2(i)=T2(i−N) Equation 1b

At 606, an average of signal strength for any read element j is obtained to obtain T1ₐᵥ. The averaging can take into account the signal strength for multiple preceding read elements, for example.

Similarly, at 607, an average of signal strength for any read element j is obtained to obtain T2ₐᵥ. The averaging can take into account the signal strength for multiple preceding read elements, for example.

Example graphical illustrations of T1ₐᵥ, T2ₐᵥ are provided in FIG. 5a, with the averaging being conducted at least at a first time point t₁ and a second time point t₂ and, in some embodiments, for all read elements after the initial time point.

At 608, calibration and normalisation in relation to the T1ₐᵥ values is carried out to obtain T1ₙₒᵣₘ. In the normalisation procedure, the T1ₐᵥ values are normalised based on T1ₚₑₐₖ being adjusted to a normalisation value such as 1 or 100, following subtraction of the dry read measurement T1_{dry} from both the T1ₐᵥ values and the peak value T1ₚₑₐₖ.

At 609, calibration and normalisation in relation to the T2ₐᵥ values is carried out to obtain T2ₙₒᵣₘ. In the normalisation procedure, the T2ₐᵥ values are normalised based on T2ₚₑₐₖ being adjusted to the same normalisation value as used for T1ₐᵥ (e.g. 1 or 100), following subtraction of the dry read measurement T2_{dry} from both the T2ₐᵥ values and the peak value T2ₚₑₐₖ.

Example graphical illustrations of T1ₙₒᵣₘ and T2ₙₒᵣₘ are provided in FIG. 5b. As can be seen by comparing FIG. 5b with FIG. 5a, the T1 and T2 signals in FIG. 5b have been time shifted to account for the temporal lag, and normalised based on matching of the T1ₚₑₐₖ and T2ₚₑₐₖ values.

At 610, a delta value Δi is computed at a first time point, t=t₁ minutes, from detection of conjugate front at T1 (i.e. from the initial time point). t₁ may be 3 minutes, for example. This delta value is indicative of the divergence in signal strength between the T1 and T2 channels at the first time point. In a negative test, where there is little or no immobilization of first labelled complexes at any test zone, it would be expected that the delta value Δi is very low or zero. In a positive test, where there is immobilization of first labelled complexes at one of the test zones but not the other, it would be expected that the delta value Δi is relatively substantial, as represented in FIG. 5b.

At 611, a delta value Δf is computed at a second time point, t=t₂ minutes, from detection of conjugate front at T1. t₁ may be 6 minutes, for example. This delta value is indicative of the divergence in signal strength between the T1 and T2 channels at the second time point. In a negative test, where there is little or no immobilization of first labelled complexes at any test zone, it would be expected that the delta value Δf is very low or zero. In a positive test, where there is immobilization of first labelled complexes at one of the test zones but not the other, it would be expected that the delta value Δf is relatively substantial and will have increased over the delta value Δi, as represented in FIG. 5b.

At 612, an S value is calculated by comparing Δi and Δf. Calculation of the S value is represented in Equation 2 below.

S=Δf−Δi Equation 2

A test value such as the S value (which may be positive or negative depending on which of the first and second test zones immobilizes the analyte of interest, for example) can be used to make a determination about a medical condition of a human or animal body from which the sample was received. For example, if the S value is within a nominal threshold range the determination of the medical condition can be assigned a negative test outcome (e.g. “no flu”). An S value that is exceeds the threshold (whether by being below a lower bound of the normal range or above a higher bound of the normal range) can be assigned as a positive test.

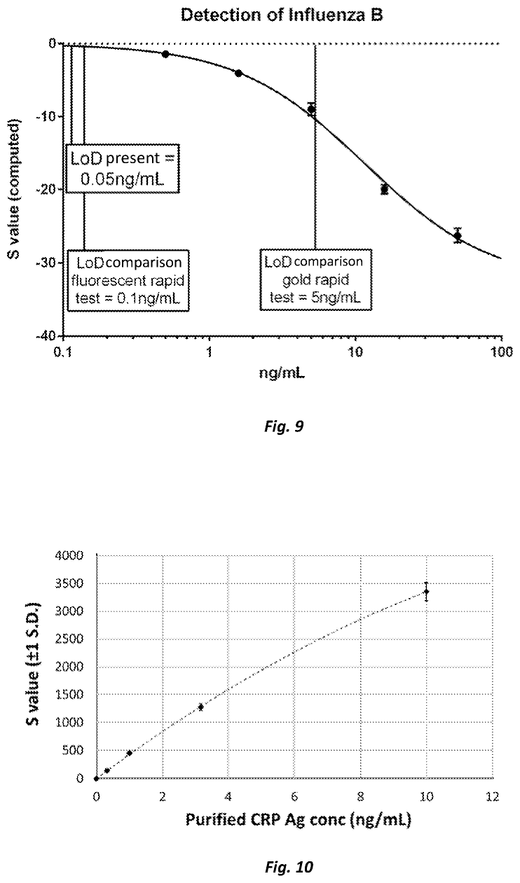

By eliminating the intrinsic variability of the conventional “peak clearance” approach exemplified in FIG. 4, for example, the “accretion method” of the present disclosure, exemplified in FIGS. 5a and 5b, for example, can deliver high reproducibility. Accordingly, calibration curves or look-up tables can be developed for each target analyte, where a test value (e.g. an S value) can be reliably correlated with a specific target analyte concentration in the sample. FIG. 9 provides a graph illustrating a correlation of S value with analyte concentration calculated using the accretion method according to embodiments of the present disclosure, for multiple test samples. Each sample had different amounts of recombinant Influenza B nucleoprotein added therein. The data indicates that the S value correlates with the analyte concentration, with linear response across a dynamic range of approximately 2 logs. The measurements are highly reproducible, with CV<10% (n=6 independent repeats). The linear response and small value of the CV of the measurement enables estimation of the antigen concentration in the sample through inspection of the S value (e.g. a value of S=−20 corresponds to approximately 20 ng/mL of Influenza B nucleoprotein). The linear response can provide for a significantly lower limit of detection (LoD) using the present accretion method (e.g. 0.05 ng/ml) in comparison to conventional tests using fluorescent labels (0.1 ng/L) or gold particles (5 ng/ml).

Whilst qualitative detection of a biomarker is sufficient for certain illnesses (e.g. influenza), where the titre does not necessarily correlate with severity of the disease, antigen quantitation can be essential in some situations. One example is C-reactive protein, which is a nonspecific marker of inflammation and used to assess the onset of an infection. FIG. 10 provides a graph illustrating a correlation of S value with analyte concentration for a purified CRP antigen diluted in suitable assay buffer. The different concentrations (four replicates at each concentration with a CV of <6%) represented a 1000-fold dilution of serum in the clinically relevant range for a high-sensitivity CRP assay (i.e. <1 to 10 mg/L). The results demonstrated that quantitative and rapid detection (e.g. within 8 minutes or less from sample loading) were possible using the accretion method according to embodiments of the present disclosure.

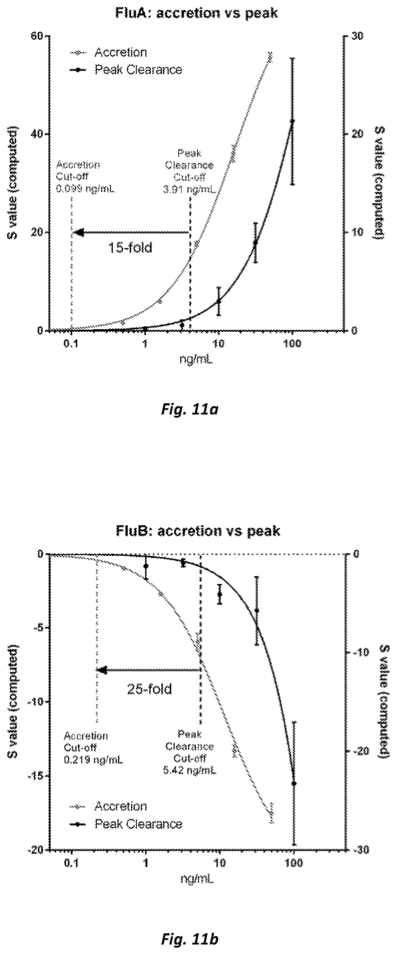

The accretion method according to embodiments of the present disclosure can deliver higher sensitivity when directly compared with the conventional test assays based on peak clearance. FIGS. 11a and 11b provide graphs illustrating the performance of the accretion method vs the conventional peak clearance method where the target analyte is Influenza A antigen and Influenza B antigen, respectively, the reagents and the antigen dilutions being the same for the two assay formats. Cut-off values (indicative of assay sensitivity) for both the accretion method and conventional peak clearance approach have been calculated by estimating the average of n=6 blank measurements plus three times the standard deviation of the measurements. Assuming that the distribution of the measurements follows a Gaussian distribution, a value of three standard deviations from the average corresponds to 99.7% of the measurements occurring within the range (e.g. 0.3% false results). The accretion method delivers a 15-fold (Influenza A) and 25-fold (Influenza B) gain in sensitivity compared to the conventional approach. In addition, the intrinsic variability in the fluidic profile of the conventional approach results in highly variable results, with CV>20-30% in certain conditions. Conversely, the accretion method delivers CV<10% consistently, and in most instances the CV<5%. This can be important when attempting to accurately quantify the concentration of the antigens in the sample.

As described in further detail below, in some embodiments of the present disclosure, the methods and apparatus may be capable of making a determination about two or more different analytes of interest. The presence of either one of the two analytes of interest in the sample may be mutually exclusive of the presence of the other analyte of interest, or otherwise. So that the methods and apparatus can account for the presence of two or more analytes of interest, the sample may also be incubated with at least a second immobilised capture reagent comprising labels, wherein the second mobilisable capture reagent is able to bind specifically to the second analyte of interest in the sample to form a plurality of second labelled complexes. When determinations about two analytes of interest are made, two test zones may still be used. Where determinations about three of more analytes of interest are made, three or more test zones may be used in the lateral flow device.

Thus, the methods and apparatus of the present disclosure may make determinations about a plurality of different analytes in the sample and selectively indicate to the user the presence of one of a plurality of medical conditions, based on identification of one of the different analytes.

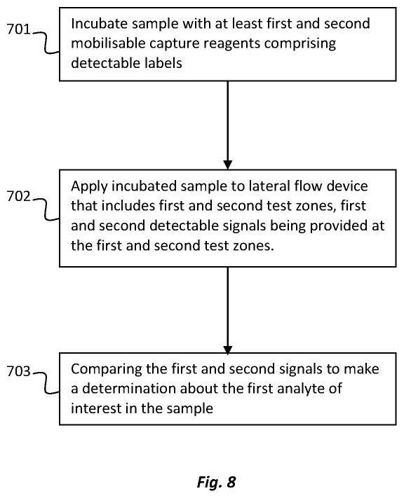

In one embodiment, as illustrated in the flowchart of FIG. 8, at 701, a sample is incubated with at least a first mobilisable capture reagent comprising detectable labels and a second mobilisable capture reagent comprising detectable labels. During the incubation, the first mobilisable capture reagent is able to bind specifically to a first analyte of interest, if present in the sample, to form a plurality of first labelled complexes, and the second mobilisable capture reagent is able to bind specifically to a second analyte of interest, if present in the sample, to form a plurality of second labelled complexes.

At 702, after the incubating is carried out, the sample (as a post-incubation mixture) is applied to a lateral flow device that includes first and second test zones, e.g. as illustrated in FIG. 1. Any first labelled complexes and any second labelled complexes in the sample can provide for detectable first and second signals at the first and second test zones

In this embodiment, one of the first and second test zones is configured to immobilize a plurality of the first labelled complexes, but not the second labelled complexes, and the other of the first and second test zones is configured to immobilize a plurality of the second labelled complexes, but not the first labelled complexes.

At 703, after the initial time point when the front of the sample arrives at the first and second test zones, the first and second signals are compared to make a determination about both the first analyte of interest in the sample and the second analyte of interest in the sample. The comparison process can be identical to the process described above, with reference to FIGS. 5a to 7, for example. In this process, to the extent that the presence of one of the first and second analytes of interest in the sample is mutually exclusive of the presence of the other, the presence and level of either analyte in the sample will be differentiated by whether the delta values Δi and Δf, and the S value, are positive or negative.

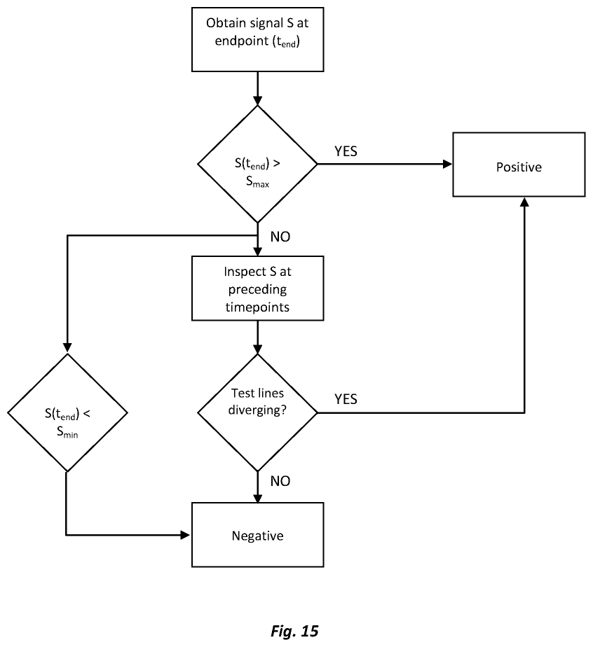

As discussed above, a ‘positive’ test (i.e. presence of the medical condition) can be identified if the S value exceeds a threshold value (Sₘₐₓ). For example, where an S value is obtained at the endpoint of testing a positive test may be identified if S(t_{end})>Sₘₐₓ. However, with reference to the decision making flow chart illustrated in FIG. 15 a positive test can also identified by the reader, even if the S value does not exceed the threshold value at the test end point (t_{end}), if a progression of determined S values up to the endpoint of testing (t_{end}) indicate that a subsequent S value would, in due time, exceed the threshold value. For example, for successive time periods (t₁, t₂, t₃ . . . ) up to the test end point (t_{end}) a positive test may be identified if test values are continuously increasing, e.g. S(t₁)<S(t₂)<S(t₃) . . . . The advantage of this approach is illustrated with reference to FIG. 14, which shows calibrated and normalised first and second signal levels T1ₙₒᵣₘ, T2ₙₒᵣₘ in which signal T2ₙₒᵣₘ is progressively increasing (and therefore diverging from signal T1ₙₒᵣₘ) but only by a small amount (e.g. in comparison to the signals of FIG. 5b). Nevertheless, the decision flow may include a restriction, such as a minimum level Sₘₐₓ that the S value at the endpoint of testing (t_{end}) must exceed if any positive test is to be identified.

Any reader or processor used in the present disclosure may comprise one or more processors and data storage devices. The one or more processors may each comprise one or more processing modules and the one or more storage devices may each comprise one or more storage elements. The modules and storage elements may be at one site, e.g. in a single hand-held device, or distributed across multiple sites and interconnected by a communications network such as the internet.

The processing modules can be implemented by a computer program or program code comprising program instructions. The computer program instructions can include source code, object code, machine code or any other stored data that is operable to cause a processor to perform the methods described. The computer program can be written in any form of programming language, including compiled or interpreted languages and can be deployed in any form, including as a stand-alone program or as a module, component, subroutine or other unit suitable for use in a computing environment. The data storage device may include suitable computer readable media such as volatile (e.g. RAM) and/or non-volatile (e.g. ROM, disk) memory or otherwise.

The lateral flow device or lateral flow assay in accordance with one or more embodiments of the present disclosure may operate as a single unit. For example, the device or assay may be provided in the form of a hand-held device. The device or assay may be a single-use, disposable, device. Alternatively, the device or assay may be partly or entirely re-usable. While in some embodiments the device or assay may be implemented in a laboratory, the apparatus may designed as a ‘point-of-care’ device, for home use or use in a clinic, etc. The device or assay may provide a rapid-test device, with identification of target conditions being provided to the user relatively quickly, e.g., in under 10 minutes.

The apparatus of one or more embodiments of the present disclosure may be configured for use with a variety of different types of biological samples. The sample may be a fluid sample. Biological samples which may be used in accordance with the apparatus and/or method of one or more embodiments of the present disclosure include, for example, saliva, mucus, blood, serum, plasma, urine, vaginal discharge and/or amniotic fluid. A biological sample which may be used in accordance with the apparatus and/or method of one or more embodiments of the present disclosure is saliva, mucus or other respiratory aspirate.

The lateral flow device or assay of one or more embodiments of the present disclosure may be used in a method of determining whether or not a subject is infected with one or more pathogens e.g., Influenza virus. The methods may be carried out in a home environment or in a laboratory setting, or other environment. The methods may comprise using an apparatus of an embodiment as disclosed herein.

At least the first analyte may be one or more specific biological entities, such as one or more antigens. For example, the antigens may be from one or more respiratory or blood-borne viruses including, but not limited to, Influenza A (including the H1N1 virus subtype), Influenza B, Respiratory Synctial Virus, parainfluenza viruses, adenoviruses, rhinoviruses, coronaviruses, coxsackie viruses, HIV viruses, and/or enteroviruses. The apparatus and methods may also be used to test for sexually transmitted infections, such as bacterial infections known to spread by sexual contact (e.g., gonorrhoea, chlamydia or otherwise), and viral infections known to spread by sexual contact (e.g., herpes simplex viruses (HSV), papillomaviruses (HPV), human immunodeficiency virus (HIV), hepatitis B virus, and cytomegalovirus). In such examples, the antigens will be from one or more pathogens which cause the sexually transmitted infection or disease. Nonetheless, a wide variety of other medical conditions based on viruses, infections or otherwise may be tested using apparatus and methods according to the present disclosure.

The lateral flow assay or lateral flow device of one or more embodiments of the present disclosure may be provided in a kit. In one example, a kit may comprise the lateral flow assay or device of an embodiment of the present disclosure and instructions for use. The instructions for use may provide directions for using the assay or device to determine whether or not a subject is infected with one or more pathogens e.g., influenza virus, in accordance with a method of the present disclosure. In each of the examples, the kit may optionally comprise one or more incubation vessels configured for the particular diagnostic application of interest.

As described herein, the lateral flow device may be configured to include one or more capture reagents. Capture reagents used in accordance with one or more embodiments of the present disclosure may be any one or more agents having the capacity to bind an analyte of interest in a sample. The capture reagent may be configured to bind with specificity to a particular analyte. In accordance with one example, the capture reagents may have the capacity to bind with specificity to a virus antigen to form a binding pair or complex. However, the device may be configured to include capture reagents having the capacity to bind, and form a binding pair or complex with, antigens from other infectious pathogens as required for the particular diagnostic application. Some examples of such binding pairs or complexes include, but are not limited to, an antibody and an antigen (wherein the antigen may be, for example, a peptide sequence or a protein sequence); complementary nucleotide or peptide sequences; polymeric acids and bases; dyes and protein binders; peptides and protein binders; enzymes and cofactors, and ligand and receptor molecules, wherein the term receptor refers to any compound or composition capable of recognising a particular molecule configuration, such as an epitopic or determinant site.

The term “immobilised”, as used with respect to a capture reagent, means the reagent is attached to one of the test zones of the lateral flow device such that lateral flow of the sample through or along the absorbent pad material of the lateral flow device during an assay process will not dislodge the reagent. The capture reagent may be immobilised by any suitable means known in the art. Conversely, the terms “mobilisable” is used to indicate that the capture reagent is capable of moving with the sample, either by itself or as part of a complex comprising the capture reagent and cognate analyte, through the lateral flow device from at least the receiving portion to the test portion, and as an example, a capture reagent which binds specifically to an influenza A virus antigen may not bind significantly or at all to any other analytes or components in a sample, such as an influenza B virus antigen, if present in the sample.

In accordance with one particular example, the or each capture reagent is an antibody or an antigen binding portion thereof. The skilled person will be aware that an “antibody” is generally considered to be a protein that comprises a variable region made up of a plurality of immunoglobulin chains, e.g., a polypeptide comprising a V_{L} and a polypeptide comprising a V_{H}. An antibody also generally comprises constant domains, some of which can be arranged into a constant region or constant fragment or fragment crystallizable (Fc). A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG1, IgG2, IgG3, IgG4, IgA₁ and IgA₂) or subclass. The term “antibody” also encompasses humanized antibodies, human antibodies and chimeric antibodies. As used herein, the term “antibody” is also intended to include formats other than full-length, intact or whole antibody molecules, such as Fab, F(ab′)2, and Fv which are capable of binding the epitopic determinant. These formats may be referred to as antibody “fragments”. In accordance with one or more embodiments in which the device 110 of the disclosure includes an antibody fragment configured to detect an influenza virus antigen, it will be expected that antibody fragments retain some or all of the ability of the corresponding full-length, intact or whole antibody to bind to the influenza virus antigen, as required. Examples of antibody fragment formats which retain binding capability include, but are not limited to, the following:

(1) Fab, the fragment which contains a monovalent binding fragment of an antibody molecule and which can be produced by digestion of whole antibody with the enzyme papain to yield an intact light chain and a portion of one heavy chain;
(2) Fab′, the fragment of an antibody molecule which can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, defined as a genetically engineered fragment containing the variable region of the light chain and the variable region of the heavy chain expressed as two chains;
(5) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, and tetrabodies etc which may or may not be polyspecific (see, for example, WO 94/07921 and WO 98/44001); and
(6) Single domain antibody, typically a variable heavy domain devoid of a light chain.

Accordingly, an antibody used as a capture reagent in accordance with one or more embodiments of the present disclosure may include separate heavy chains, light chains, Fab, Fab′, F(ab′)₂, Fc, a variable light domain devoid of any heavy chain, a variable heavy domain devoid of a light chain and Fv. Such fragments can be produced by recombinant DNA techniques, or by enzymatic or chemical separation of intact immunoglobulins.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including a Fc region. The constant domains may be wild-type sequence constant domains (e.g., human wild-type sequence constant domains) or amino acid sequence variants thereof. In some cases, the intact antibody may have one or more effector functions.

An antibody used as a capture reagent in accordance with one or more embodiments of the present disclosure may be a humanized antibody. The term “humanized antibody”, as used herein, refers to an antibody derived from a non-human antibody, typically murine, that retains or substantially retains the antigen-binding properties of the parent antibody but which is less immunogenic in humans.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method of performing a lateral flow test for making a determination about at least a first analyte of interest in a sample from a body, the method comprising:
applying a sample to a receiving portion of a lateral flow device such that the sample flows from the receiving portion to at least a first test zone and a second test zone of the lateral flow device,
monitoring levels of first and second signals at the first and second test zones over an assay period wherein, if the first analyte of interest is present in the sample, the first analyte is labelled and wherein the presence of labelled first analyte in the sample causes the level of one of the first and second signals to increase during the assay period; and
monitoring a change between the first and second signal levels over a period of time during the assay period.

2. The method of claim 1 comprising identifying a baseline level of the first and second signals prior to an initial time point at which a front of the sample arrives at the first and/or second test zone from the receiving portion, and subtracting the baseline level from the first and second signal levels to obtain calibrated first and second signal levels after the initial time point.

3. The method of claim 1 comprising normalising the first and second signal levels.

4. The method of claim 2 comprising normalising the calibrated first and second signal levels.

5. The method of claim 3 or 4, wherein the normalisation of the first and second signal levels is based on an initial signal level peak when the sample arrives at the first and second test zones.

6. The method of claim 5, wherein the normalisation of the first and second signal levels is based on a signal level that occurs after a peak signal level when the sample arrives at the first and second test zones.

7. The method of any one of the preceding claims, wherein one of the first and second test zones is further from the receiving portion than the other of the first and second test zones, and wherein the method comprises time-shifting the first and second signals to compensate for delays in the sample reaching the test zone furthest from the receiving portion.

8. The method of claim 7, wherein the monitoring of the change between the first and second signal levels is based on the first and second signals as time-shifted relative to each other.

9. The method of claim 7 or 8, wherein the time-shifting is carried out using a lag-coefficient that accounts for an increasing delay in the sample reaching the test zone furthest from the receiving portion.

10. The method of any one of the preceding claims, wherein the monitoring of the change between the first and second signal levels after the initial time point comprises determining differences between the first and second signal levels at one or more time points.

11. The method of claim 10, wherein the monitoring of the change between the first and second signal levels comprises determining differences between the first and second signal levels at least at two different time points.

12. The method of claim 10, wherein the monitoring of the change between the first and second signal levels comprises determining a difference between the first and second signal levels at least at a test end point.

13. The method of claim 10, 11 or 12, wherein the difference between the first and second signal levels at any time point is calculated as a delta value (Δ) or a ratio value (R) and wherein the monitoring of the change between the first and second signal levels comprises monitoring an evolution of the delta value (Δ) or ratio value (R).

14. The method of any one of the preceding claims, wherein the monitoring of the change between the first and second signal levels over a period of time after the initial time point comprises at least:
comparing the first and second signal levels at a first time point to obtain a signal level difference (Δi) or a ratio value (Ri) at the first time point,
comparing the normalised first and second signal levels at a second time point to obtain a signal level difference (Δf) or a ratio value (Rf) at the second time point, and
comparing the signal level difference (Δi) at the first time point with the signal level difference (Δf) at the second time point or comparing the ratio value (Ri) at the first time point with the ratio value (Rf) at the second time point.

15. The method of any one of the preceding claims, wherein the method is to make a determination about medical condition of a human or animal body based on the determination about at least the first analyte.

16. The method of claim 15, when dependent on claim 14, wherein the comparing of the signal level differences or ratio values produces a test value and wherein the determination about the medical condition is based on whether or not the test value is above or below one or more threshold values.

17. The method of claim 15, when dependent on claim 12, wherein the determining of a difference between the first and second signal levels at least at the test end point produces a test value and wherein the determination about the medical condition is based on whether or not the test value is above or below one or more threshold values.

18. The method of claim 15, when dependent on claim 11, wherein the determination of differences between the first and second signal levels at least at two different time points produces test values for the different time points and wherein the determination about the medical condition is based on whether or not the test values are following a trend.

19. The method of claim 18, wherein the trend is a continuous increase or decrease of the test values for successive time points.

20. The method of any one of the preceding claims, wherein the method is to make a quantitative determination about a level of the first analyte in the sample and/or a human or animal body providing the sample.

21. The method of any one of the preceding claims, comprising labelling the first analyte of interest in the sample prior to application of the sample to the lateral flow device.

22. The method of claim 21, wherein the labelling is performed by incubating the sample with a first mobilisable capture reagent comprising labels, wherein the first mobilisable capture reagent is able to bind specifically to the first analyte of interest, if present in the sample, to form a plurality of first labelled complexes.

23. The method of claim 21 or 22, wherein the incubating is carried out for a period of at least 10 seconds, at least 20 seconds, at least 30 seconds, at least 1 minute, at least 2 minutes, at least 5 minutes, at least 7 minutes, at least 10 minutes, at least 15 minutes, at least 20 minutes, at least 25 minutes or at least 30 minutes.

24. The method of claim 21, 22 or 23, wherein the incubating further comprises mixing the sample with a buffer solution.

25. The method of any one of claims 21 to 24, wherein the incubating is carried out by depositing the sample into the interior of a vessel, the interior of the vessel being separate from the lateral flow device.

26. The method of claim 25 wherein, prior to the depositing of the sample into the interior of the vessel, the at least a first mobilisable capture reagent is located on an inner surface of the vessel.

27. The method of any one of the preceding claims, wherein the detectable labels are fluorescent labels.

28. The method of claim 27, wherein the fluorescent labels each comprise one or more quantum dots.

29. A lateral flow assay for making a determination about at least a first analyte of interest in a sample from a body comprising:
a lateral flow device, comprising:
a receiving portion and at least first and second test zones, the receiving portion being configured to receive a sample such that the sample flows from the receiving portion to the first and second test zones,
a reader configured to:
monitor levels of first and second signals at the first and second test zones over an assay period wherein, if the first analyte of interest is present in the sample, the first analyte is labelled and wherein the presence of labelled first analyte in the sample causes the level of one of the first and second signals to increase during the assay period; and
monitor a change between the first and second signal levels over a period of time during the assay period.
